(19) United States
(12) Patent Application Publication
(10) Pub. No.: US 2023/0284342 A1
(43) Pub. Date: Sep. 7, 2023

(54) ELECTRICALLY CONDUCTIVE, POROUS SINTERING BODY

(71) Applicant: SCHOTT AG, Mainz (DE)

(72) Inventors: Dang Cuong PHAN, Aachen (DE); Matthias RINDT, Altfraunhofen (DE); Thomas BEERHORST, Altfraunhofen (DE)

(73) Assignee: SCHOTT AG, Mainz (DE)

(21) Appl. No.: 18/316,285

(22) Filed: May 12, 2023

Related U.S. Application Data
(63) Continuation of application No. PCT/EP21/82292, filed on Nov. 19, 2021.

(30) Foreign Application Priority Data
Nov. 19, 2020 (DE) 10 2020 130 559.1

(51) Int. Cl. H05B 3/18; A24F 40/10; A24F 40/46; A24F 40/44; A24F 40/70; A61M 11/04; C03C 14/00; C03C 4/14; C03C 11/00; A61L 9/03

(52) U.S. Cl. CPC H05B 3/18; A24F 40/10; A24F 40/46; A24F 40/44; A24F 40/70; A61M 11/042; C03C 14/004; C03C 4/14; C03C 11/007; A61L 9/03; H05B 2203/021; H05B 2203/022; H05B 3/12

(57) ABSTRACT

An evaporator is provided that includes a porous sintered body. The porous sintered body is formed by a composite of at least one electrically conductive material and at least one dielectric material. The sintered body has an open porosity in a range from 10 to 90% and an electrical conductivity in a range from 0.1 to 105 S/m. The fraction of electrically conductive material in the sintered body is a maximum of 90 wt. %.

### CROSS REFERENCE TO RELATED APPLICATIONS

This application is a continuation of International Application PCT/EP2021/082292 filed Nov. 19, 2021, which claims benefit under 35 USC § 119 of German Application 10 2020 130 559.1 filed Nov. 19, 2020, the entire contents of all of which are incorporated herein by reference.

### BACKGROUND

### 1. Field of the Invention

The invention relates in general to an electrically conductive porous sintered body. Specifically the invention relates to an evaporator unit comprising a liquid store, or liquid reserve, and a heating unit for the storage and regulated delivery of evaporable substances.

The evaporator unit may be used here in particular in electronic cigarettes, drug administration devices, room humidifiers and/or heatable vaporizers. These vaporizers may be apparatuses for the provision, delivery and/or propagation of substances into a gas phase, as for example into the ambient air, in the form of gases, vapors and/or aerosols. Substances used may be, for example, fragrances or active ingredients, especially insect repellants.

Electronic cigarettes, also referred to below as E-cigarettes, or similar apparatuses such as electric pipes or shishas for example, are increasingly being used as an alternative to tobacco cigarettes. The electronic cigarettes typically comprise a mouthpiece and an evaporator unit and also an electrical energy source which is in operative connection with the evaporator unit. The evaporator unit has a liquid store which is connected to a heating element.

Certain drugs, especially drugs for the treatment of respiratory tracts and/or the oral and/or nasal mucosa, are administered advantageously in a gaseous or evaporated form, as an aerosol, for example. Evaporators of the invention may be used for the storage and delivery of such drugs, especially in administration devices for such drugs.

Thermally heatable vaporizers are increasingly being used to provide fragrances to a setting. Such settings may be, in particular, bars, hotel lobbies and/or vehicle interiors, examples being the interiors of motor vehicles, especially automobiles. In the case of the evaporator unit used in that context as well, a liquid store is connected to a heating element. The liquid store contains a liquid, which is usually a carrier liquid such as propylene glycol or glycerol, for example, which comprises in solution and/or generally, adjuvants such as fragrances and flavors and/or nicotine and/or drugs. The carrier liquid is bound on the internal surface of the liquid store by adsorption processes. There may be a separate liquid reservoir provided in order to supply liquid to the liquid store.

It is generally the case that the liquid stored in the liquid store is evaporated by heating of a heating element, desorbs from the wetted surface of the liquid store, and can be inhaled by the user. Temperatures in this case may reach more than 200° C.

The liquid store or liquid reserve must therefore have a high accommodation capacity and a high adsorption effect, and at the same time the liquid at high temperatures must be rapidly delivered and transported.

Various materials are known from the prior art for use as a liquid store or wick. Hence liquid stores or wick may be formed by a porous or fibrous organic polymer. While such components are very simple to produce, there is nevertheless a risk here of the polymeric material being too highly heated and decomposing, in the wake, for example, of dry operation of the component. This not only is deleterious to the lifetime of the liquid store or wick and hence of the evaporator unit, but also carries the risk that decomposition products of the fluid for evaporation or even of the liquid store are released and inhaled by the user.

Electronic cigarettes with porous liquid stores composed of organic polymers are known from the prior art. Because of the low temperature stability of the polymeric material, therefore, there is a need to observe a minimum distance between the heating element and the liquid store. This prevents a compact construction of the evaporator unit and hence of the electronic cigarette. Alternatively to the observance of a minimum distance, a wick may be used, which guides the liquid for evaporation to the heating coil by capillary action. This wick is usually fabricated from glass fibers. While these do have a high temperature stability, the individual glass fibers can easily break. The situation is similar if the liquid store itself is also produced from glass fibers. There is therefore a risk of the user inhaling loose or solvated fiber fragments. Alternatively, wicks made of cellulose fibers, cotton or bamboo fibers may also be used. While these do have less of a fracture risk than glass fiber wicks, they are nevertheless of lower temperature stability.

Consequently, use is also made of evaporator units whose liquid stores consist of porous glasses or ceramics. Because of the higher temperature stability of these liquid stores, it is possible to realize a more compact construction of the evaporator and hence also of the electronic cigarette as a whole.

The local evaporation may be achieved in practice by a low pressure, in conjunction with a high temperature. In the case of an electronic cigarette, for example, the low pressure is realized through the suction pressure when the cigarette is drawn on during consumption and so the pressure is regulated by the consumer. The temperatures in the liquid store that are needed for the evaporation are generated by a heating unit. In this case temperatures in general of more than 200° C. are attained, in order to ensure rapid evaporation.

The heating power is usually provided by an electrical heating coil operated via single-use or rechargeable battery. The required heating power in this case is dependent on the volume to be evaporated and on the efficiency of the heating. In order to prevent decomposition of the liquid due to excessive temperatures, the heat transport from the heating coil to the liquid is to take place by contactless radiation. For this purpose, the heating coil is mounted as close as possible to the evaporation surface, but without touching it. If the coil does touch the surface, the liquid is often overheated and decomposes.

Overheating of the surface, however, may also come about in the case of heat transport by contactless radiation. The overheating usually develops locally on the surface of the evaporator opposite the heating coil. This is the case if, in operation, a large quantity of vapor is required and the transport of liquid to the surface of the evaporator is not quick enough. The energy supply from the heating element therefore cannot be utilized for the evaporation, the surface dries out, and heating may take place locally to temperatures well above the evaporation temperature, and/or the temperature stability of the liquid store is exceeded. Precise temperature adjustment and/or control is therefore vital. A disadvantage in this context, however, is the resultant complex construction of the electronic cigarette, which is manifested among other things in high production costs. Moreover, the temperature regulation may reduce the evolution of vapor and hence the maximum possible vapor intensity.

EP 2 764 783 A1 describes an electronic cigarette having an evaporator which features a porous liquid store composed of a sintered material. The heating element may take the form of a heating coil or of an electrically conducting coating, in which case the coating is deposited only on parts of the lateral faces of the liquid store. Here again, therefore, the evaporation is locally limited.

US 2011/0226236 A1 describes an inhaler wherein the liquid store and the heating element are cohesively bonded to one another. This liquid store and heating element form a flat composite material. The liquid store, composed for example of an open-pore sintered body, acts as a wick and passes the liquid for evaporation to the heating element. The heating element here is applied on one of the surfaces of the liquid store, in the form of a coating, for example. Here again, therefore, the evaporation is locally limited on the surface, with a consequent risk likewise of overheating.

In order to circumvent these problems, the prior art has disclosed evaporator units wherein the evaporation takes place not only at the surface of the liquid store but instead through its entire volume. The vapor is evolved not only locally at the surface, but instead in the entire volume of the liquid store. The vapor pressure within the liquid store is therefore largely constant and capillary transport of the liquid to the surface of the liquid store continues to be ensured. Correspondingly, the evaporation rate is no longer minimized by the capillary transport. A precondition for such an evaporator is an electrically conducting and porous material. When an electrical voltage is applied, the entire volume of the evaporator heats up, and the evaporation takes place throughout the volume.

Evaporators of this kind are described in US 2014/0238424 A1 and US 2014/0238423 A1. In these cases, liquid store and heating element are combined in one component, in the form for example of a porous body of metal or a metal mesh. A disadvantage here, however, is that in the case of the porous bodies described, the ratio of pore size to electrical resistance is not readily adjustable. In addition, following the application of the conductive coating, subsequent sintering may result in degradation of the coating.

The materials described in the above-stated prior art, however, are unsuitable or of only limited suitability for producing composite materials, by means of a sintering process, that exhibit not only a high, adjustable porosity but also good electrical conductivities. Generally speaking, ceramics, owing to their fine porosity and rough surface, are also difficult to coat comprehensively.

In DE 10 2017 123 000, therefore, there are evaporators comprising a sintered glass or glass-ceramic body whose entire surface features a conductive coating. Consequently, in contrast to the situation with sintered bodies, which have such coating only on the outer surface, evaporation occurs not only on the outer surface but also in the interior of the sintered body. In the production of such evaporators, a porous sintered glass or glass-ceramic body is generated first of all, and in a subsequent step is provided with a visibly thick conductive coating, in the form of an ITO coating, for example. A disadvantage, however, is that the production process is cost-intensive because of the high levels of conductive material required such as ITO for example. Furthermore, the subsequent application of a thick coating may adversely alter the properties of the sintered body. In particular, small pores in the sintered body may become closed by the coating, with a consequent reduction in the active surface area of the sintered body.

Also known are so-called nebulizers, which are able to atomize liquids by means of ultrasound, using piezoelectric elements, for example. The vapor or, rather, fog or mist thus generated, however, is cold, and as such is usually or often undesirable particularly in the usage of electrical cigarettes and/or medical devices.

### SUMMARY

It is an object of the invention, therefore, to provide a sintered body which in particular is suitable for use as an evaporator in electronic cigarettes and/or drug administration devices and/or thermally heated fragrance vaporizers and which does not have the disadvantages described above. The invention is therefore directed at achieving good heatability and simple adjustability of electrical resistance and porosity of the liquid store. A further object of the invention lies in the provision of a method for producing an electrically conductive sintered body of this kind.

The evaporator of the invention or evaporator unit of the invention comprises an electrically conductive porous sintered body which is embodied as a composite of at least one electrically conducting material and at least one dielectric material.

The porous evaporator uses adsorptive interactions to store a carrier liquid which may comprise, for example, fragrances and flavors and/or drugs, including nicotine and/or active ingredients in solution in suitable liquids. When an electrical voltage is applied, high temperatures arise owing to the electrical conductivity of the evaporator, and so the carrier liquid is evaporated and desorbs from the wetted surface of the evaporator, and the vapor can be inhaled by the user.

The sintered body has an open porosity in the range from 10 to 90%, preferably in the range from 50 to 80%, based on the volume of the sintered body. This gives the sintered body a large internal surface area for desorption in conjunction with a high mechanical stability and enables a good sustained flow of the liquid or medium for evaporation.

Preferably at least 90% and more particularly at least 95% of the total pore volume takes the form of open pores. This open porosity may be determined by measuring methods according to DIN EN ISO 1183 and DIN 66133. The sintered body preferably contains only a small fraction of closed pores. As a result, the sintered body has only a low dead volume, i.e., a volume which makes no contribution to the accommodation and delivery of the liquid for evaporation. The sintered body preferably has a fraction of closed pores of less than 15% or even of less than 10% of the total volume of the sintered body. For determining the fraction of the closed pores, it is possible to determine the open porosity as described above.

The total porosity is computed from the density of the body. The fraction of closed pores is then the result of the difference between total porosity and open porosity. According to one embodiment of the invention, indeed, the sintered body has a fraction of closed pores of less than 5% of the total volume, these pores possibly occurring due to the process.

As dielectric material the sintered body comprises at least one material selected from the group of glass, glass-ceramic, ceramic and combinations thereof. According to one embodiment, the sintered body comprises at least two different dielectric materials. In particular, the dielectric materials used have no significant electrical conductivity at room temperature. Dielectric material and electrically conductive material here form the composite material of the sintered body. A dielectric or dielectric material for the purposes of the present disclosure refers in particular to a substance having weak or no electrical conduction, in which the charge carriers present are not freely mobile, or at least not freely mobile at room temperature.

The fraction of dielectric material is at least 10 vol %, with one embodiment of the invention envisaging a fraction of dielectric material in the composite material in the range from 30 to 95 vol %. The fraction of electrically conductive material in the composite material is at most 90 vol %. According to one embodiment of the invention, the fraction of electrically conductive material in the composite material is 5 to 70 vol %, preferably 10 to 60 vol %, most preferably 15 to 40 vol %. The fractions recited above are based here on the composite material of the sintered body, meaning that in this case the pore volume or volume fraction of the pores in the sintered body is disregarded.

The sintered body of the invention, surprisingly, exhibits good electrical conductivity even with relatively low fractions of electrically conductive material. Hence according to one refinement, the sintered body contains the conductive material at not more than 40 vol % or even at most 30 vol % or even at most 20 vol %. This provides the possibility of a sintered body having an adjustable electrical conductivity in the range of the invention with a high mechanical strength at the same time. The amount of electrically conductive particles used in each case is dependent here on the particular material of the electrically conductive particles, especially on their electrical conductivity and also on the shape of the particles used. Having emerged as being particularly advantageous in this context are sintered bodies whose fraction of electrically conductive particles is at least 5 vol %, preferably at least 10 vol % and more preferably at least 15 vol %.

According to one embodiment of the invention, the amount of electrically conductive particles in the sintered body is 10 to 40 vol %, preferably 15 to 25 vol %.

Surprisingly, however, even in the case of low amounts of electrically conductive material, it is possible to attain the electrical conductivity of the sintered body in accordance with the invention. According to one further embodiment, the fraction of electrically conductive material is only 10 to 20 vol %.

Depending on the dielectric material used and on the fraction of electrically conductive material in the sintered body, this body, in spite of the low fraction of electrically conductive material, exhibits an electrical conductivity. It is thought that, owing to its homogeneous distribution in the sintered body, the electrically conductive material in the sintered body of the invention, even in relatively low amounts, forms scaffolds or three-dimensional networks of the electrically conductive material in the dielectric material, with these scaffolds or networks enabling the flow of current through them.

It is additionally thought that the flow of current can also take place as a result of electron tunneling effects. The portion of the current flow which takes place as a result of these electron tunneling effects, as a proportion of the overall electrical conductivity, increases as the amount of electrically conductive particles in the sintered body goes down.

According to one preferred embodiment, the material of the electrically conductive particles has a resistance with a positive temperature coefficient. This facilitates the regulation of the electrical heating of the sintered body and supports rapid heating starting from room temperature. Good regulatability is also provided, in an alternative or additional embodiment, if the temperature coefficient of the electrical resistance is close to zero, more particularly amounting to less than 0.00025 K⁻¹. This is the case, for example, with some copper-nickel alloys, such as Konstantan®. Konstantan has a temperature coefficient of −0.000074 K⁻¹. It is also possible to use NiCr80, with a temperature coefficient of +0.00011 K⁻¹.

In one embodiment of the invention, the maximum distance between two adjacent electrically conductive particles is less than 30 μm or even less than 10 μm. As a result of this small distance between the electrically conductive particles, the flow of current can take place by electron tunneling effects. According to one refinement of this embodiment, the electrically conductive particles are at least partly distanced from one another. In this case the electrically conductive particles are insulated from one another by the dielectric material and/or pores. Having emerged as being particularly advantageous is a mean distance between adjacent electrically conductive particles in the range from 1 to 30 μm, preferably in the range from 1 to 10 μm.

The higher the electrical conductivity of the material used in each case, the lower can be the amount of electrically conductive particles. In the case of a relatively low filling level of the electrically conductive particles in the sintered body in turn, particularly high strengths can be achieved.

The electrically conductive material is present in particulate form, while the dielectric material forms a matrix for the electrically conductive particles. The composite material of the sintered body is therefore composed of a dielectric matrix with electrically conductive particles embedded therein. The electrically conductive particles here are distributed homogeneously in the sintered body. The distribution of the conductive particles in a matrix of dielectric material ensures that the sintered body has an electrical conductivity in the range from 0.1 to 10⁵ S/m. The sintered bodies of the invention therefore have a significantly lower electrical conductivity than metallic sintered bodies known from the prior art or corresponding composite materials having higher metal contents. According to one embodiment of the invention, the electrical conductivity of the sintered body is in the range from 10 to 10000 S/m. The conductivity values are valid in particular at room temperature.

The electrical conductivity of the sintered body in accordance with the invention allows the corresponding evaporator to be used, for example, in an electronic cigarette or corresponding apparatuses such as electric pipes or shishas, for example. Thus, according to one refinement of the invention, the sintered body has an electrical resistance in the range from 0.05 to 5 ohms, preferably from 0.1 to 5 ohms. In this refinement, the evaporator is operated with a voltage in the range from 1 to 12 V and/or with a heating power of 1 to 500 W, more particularly with a heating power in the range from 1 to 300 W, preferably in the range from 1 to 150 W. In this case the evaporator heats up throughout its volume in response to the application of a current, and so the desorption of the liquid stored in the evaporator commences.

In contrast to this, apparatuses according to a different refinement may also be operated at voltages of 110V, 220V/230 V or even 380 V. Here, electrical resistances of up to 3000 ohms and powers of up to 1000 W or more are advantageous. According to one embodiment of this refinement, the apparatus in question comprises inhalers for the medical sector.

Depending on the particular use of the evaporator unit, it may have higher operating voltages, more particularly operating voltages in the range from >12V to 110 V, resistances of more than 5 ohms and/or heating powers of more than 80 W. According to one embodiment of this refinement, the apparatus comprises inhalers for the medical sector. The evaporator apparatuses of this refinement may also be designed for evaporation in larger settings, as a smoke machine, for example.

The entire accessible surface of the sintered body consisting of composite material in this case forms the evaporation face. As a result of the electrical conductivity of the sintered body in accordance with the invention, the flow of current takes place over the entire body volume of the sintered body. Correspondingly, the liquid for evaporation is evaporated on the entire surface of the sintered body. The vapor is therefore formed not only locally on the lateral face of the sintered body, but also on the internal surface of the sintered body.

In a departure from the case with evaporators with a local heating facility, a heating coil for example, or an electrically conducting coating on the lateral faces of the evaporator body, a capillary transport from the interior of the sintered body to a local heating facility is not necessary, i.e., over relatively long distances, not necessary, since in the case of the evaporator of the invention its entire volume is heated. This prevents the evaporator running dry if the capillary effect is too low and so also prevents local overheating. This has advantageous consequences for the lifetime of the evaporator unit. Moreover, in the event of local overheating of the evaporator, there may be processes of decomposition of the liquid for evaporation. On the one hand this may be problematic, since, for example, the active ingredient content of a drug for evaporation is reduced accordingly. On the other hand, decomposition products are inhaled by the user, and this may harbor health risks. In the case of the evaporator of the invention, in contrast, this danger is much lower.

The relatively high fraction of dielectric material in the sintered body results in the sintered body having good mechanical stability and strength. The use of a sintered body in the form of a composite, i.e. of a sintered body wherein dielectric material and electrically conductive particles are in homogeneous or at least largely homogeneous distribution, in contrast to retrospectively coated sintered bodies, offers the advantage that there are no disadvantageous effects on properties of the sintered body, such as, for example, its pore size or the fraction of open pores in the sintered body.

Metals in particular have emerged for use as electrically conductive material in the sintered body. According to one alternative or additional embodiment, the electrically conductive material used comprises a material having an electrical resistance with positive temperature coefficient.

Having emerged as being particularly advantageous is the use of metals with high electrical conductivities such as precious metals, copper, tungsten, molybdenum, aluminum and corresponding alloys or mixtures thereof, stainless steel or else materials such as titanium, nickel, chromium, iron, steel, manganese, silicon and graphite and corresponding alloys, such as typical heat conductor alloy, more particularly CuMnNi alloys (e.g., Konstantan®) or FeCrAl alloys (e.g., Kanthal®) or mixtures thereof. According to one embodiment the electrically conductive material used comprises a heat-resistant, preferably stainless steel, of type 1.4828 or 1.4404, for example. It has emerged as being particularly advantageous to use electrically conductive materials, more particularly metals, which have a temperature coefficient of the electrical resistance of >−0.075 l/K, but preferably ≥−0.0001 l/K, more preferably ≥0.0001 l/K. According to one advantageous configuration, the electrically conductive material here has a temperature coefficient of the electrical resistance of <0.008 l/K.

According to one advantageous configuration of the invention, the electrically conductive material in the sintered body comprises precious metals, more particularly platinum, gold, silver or their alloys or mixtures thereof.

As well as a high electrical conductivity, precious metals additionally afford the advantage that even at high temperatures they are inert or at least largely inert toward the constituents of the dielectric material, thus more particularly being materials which have little or no tendency toward reactions with the dielectric material and/or toward formation of oxide, or other chemical alteration. Inertness is therefore also an important criterion for the selection of other electrically conductive materials and/or their alloys and/or mixtures, apart from the precious metals and/or their alloys and/or mixtures. This is particularly advantageous in the context of embodiments wherein glasses are employed as dielectric material. Alternatively or additionally it is possible to use carbon as electrically conductive material, more particularly in the form of graphene, graphite or nanotubes or nanorods.

A classification of the electrically conductive materials may be performed accordingly on the basis in particular of the electrical conductivity.

The following subdivision is performed in particular:

| Class | Example | electrical conductivity/S/μm |
|---|---|---|
| A | Ag, Cu, Au, Al | more than 30 |
| B | W, Mo, Zn, Fe, Pt, Ni | 10 to 30 |
| C | Ti, Cr, steel, C, Mn, Si | less than 10 |

The invention uses electrically conductive materials having a volume fraction of at most 90 vol % in the sintered body. The fraction of the respective material in the composite is adapted here preferably to the electrical conductivity of the material used. Depending on the electrical conductivity of the material used from classes A, B, C or a mixture thereof, it is possible to vary the necessary volume fraction thereof in order to achieve a required electrical conductivity on the part of the sintered body.

In one variant, therefore, the electrically conductive material has an electrical conductivity in the range from more than 30 to 70 S/μm. In this refinement, therefore, the electrically conductive materials used comprise, in particular, silver, copper, gold and/or aluminum. Owing to the relatively high electrical conductivity, it is possible to reduce the fraction of the electrically conductive material in the composite. In one embodiment, therefore, the fraction of electrically conductive material is 5 to 40 vol %, preferably 10 to 30 vol %, more preferably 15 to 25 vol %.

According to another variant, electrically conductive materials having an electrical conductivity in the range from 10 to 30 S/μm are used, more particularly tungsten, molybdenum, zinc, iron, platinum and/or nickel. The amount of electrically conductive material is 10 to 60 vol %, preferably 15 to 50 vol %, more preferably 20 to 40 vol %.

In still another variant, an electrically conductive material is used which has an electrical conductivity in the range of, for example, 1 to less than 10 S/μm, more particularly titanium, manganese, chromium, steel, silicon and/or carbon. In this variant, according to one embodiment, the fraction of electrically conductive material is 15 to 90 vol %, preferably 20 to 70 vol %, more preferably 25 to 60 vol %.

Generally, the conductivity values stated here refer to the value thereof at room temperature.

The electrical conductivity of the sintered body may be influenced not only by the electrical conductivity of the electrically conductive material used in each case and also by the amount thereof in the sintered body, but also via the particle size of the electrically conductive particles and also by the particle shape or particle geometry. Hence in particular the use of electrically conductive particles which deviate from the round grain shape, i.e., substantially spherical particles, has emerged as being advantageous. According to one embodiment, therefore, the electrically conductive particles have a flat, platelet-shaped form and are also referred to as platelets. Alternatively or additionally, the composite comprises electrically conductive particles having a long-grained or elongate geometry. More particularly these particles have an acicular geometry. Mixtures of one or more of these grain shapes as well have emerged as being particularly advantageous. In contrast to spherical particles, for example, platelet-shaped or elongate particles are able, even at relatively low filling levels, to form a continuous scaffold of electrically conductive material within the sintered body, and so the corresponding sintered body, in spite of a relatively low filling level of the electrically conductive material, has an electrical conductivity within the range according to the invention. Accordingly, a required electrical conductivity on the part of a sintered body can be achieved in the case of elongate electrically conductive particles with a lower volume fraction than with spherical particles. Further possibilities for reducing this volume component, including with respect to elongate particles, often likewise hand in hand with further-reduced costs, may be achieved by means of platelet-shaped particles.

Furthermore, the use of flat, platelet-shaped or elongate electrically conductive particles is also especially advantageous when the filling level of the electrically conductive material in the sintered body is relatively low. By means of electrically conducting particles having the above-described geometries, it is possible in this case, even at low filling levels, to form a scaffold or network of electrically conductive material in the sintered body, and so an electrical conduction can be ensured and, when a voltage or current flow is applied through the sintered body of suitable size, the use thereof as a heating element and/or in an evaporator, for example, is made possible.

According to one embodiment of the invention, the sintered body comprises electrically conductive particles having a platelet-shaped or elongate geometry. In a refinement of the invention, the electrically conductive particles have a maximum thickness dₘₐₓ and a maximum length lₘₐₓ, for which dₘₐₓ<lₘₐₓ. Having emerged as being particularly advantageous are electrically conductive particles for which 2 dₘₐₓ<lₘₐₓ, preferably 3 dₘₐₓ≤; lₘₐₓ, more preferably 7 dₘₐₓ<lₘₐₓ.

According to one refinement of the invention, the electrically conductive particles in the sintered body have a mean particle size (d₅₀) in the range from 0.1 μm to 1000 μm, preferably in the range from 1 to 200 μm, most preferably from 1 to 50 μm. When using electrically conductive particles having a lower particle size, it is necessary to increase the fill level of the electrically conductive particles in the corresponding sintered bodies in order to achieve sufficient electrical conductivity. Hence the electrical conductivity is lowered through the use of very small electrically conductive particles. Electrically conductive particles that are too large, for their part, may greatly lower the electrical resistance in local regions of the sintered body, so making the sintered body uneven in respect of the electrical resistance. This may lead in turn to local overheating in the sintered body and to an uneven evaporation. The greater the electrical conductivity of the electrically conductive particles in question, the greater the extent to which this effect is pronounced. Moreover, very large electrically conductive particles and the associated uneven structure of the sintered body may have adverse consequences for its mechanical strength.

According to one embodiment of the invention, the pores have a mean pore size in the range from 1 μm to 1000 μm. The pore size of the open pores of the sintered body is preferably in the range of 50 to 800 μm, more preferably in the range from 100 to 600 μm. Pores with such sizes are advantageous because they are small enough to generate sufficiently great capillary force and so to ensure the replenishment of liquid for evaporation, particularly in the context of use as a liquid store in an evaporator; at the same time, they are large enough to enable rapid delivery of the vapor. In this context it is also conceivable to provide, advantageously, more than one pore size or more than one pore size range, such as a bimodal pore size distribution with large pores and small pores, for example, in a sintered body. It has additionally emerged that for a mandated or required electrical conductivity of a sintered body, the fraction of electrically conductive particles can be lower in the case of low porosity than in the case of sintered bodies of higher porosity. The respective use and the requirements thereof, as described above, such as the transport of a liquid for evaporation as against the evaporation power, for example, can therefore be honored by means of suitable adaptations to the porosity and composition of the material. The dielectric material in the sintered body is preferably thermally stable with respect to temperatures of at least 300° C. or even of at least 400° C.

According to one embodiment of the invention, the dielectric material of the sintered body comprises a glass. In one embodiment here, the amount of glass in the sintered body is at least 5 vol %. According to a further embodiment, however, only a small glass fraction of less than 5 vol % may also be provided, for instance in order to bind other particles—ceramic particles for example. According to one embodiment, the matrix of the sintered body in which the electrically conductive particles are embedded is formed of glass. The use of glass as dielectric material is advantageous in light of the processability during the production of a sintered body and also in light of the temperature stability and the mechanical strength of the glass. Having emerged as being particularly advantageous in this context are glasses without or with a relatively low alkali metal content. Alkali-free glasses, or glasses without alkali metal content, are understood here to be glasses where alkali metals are not deliberately added to their composition. Low alkali metal fractions, introduced into the glass in the form of impurities, for example, are not ruled out, however. A low alkali metal content, more particularly a low sodium content, is advantageous here from a number of standpoints. For instance, glasses having a relatively low alkali metal content exhibit low alkali metal diffusion even at high temperatures, and so there is no alteration or virtually no alteration to the properties of the glass even during hot operation of the evaporator. The low level of alkali metal diffusion on the part of the glasses is also advantageous, moreover, in the operation of the sintered body as an evaporator, since there is therefore no interaction of any such constituents—possibly emerging constituents—with the electrically conductive material and/or with a coating optionally present on the sintered body and/or with the liquid for evaporation. The latter advantage is relevant particularly in the context of the use of the optionally coated sintered body as an evaporator in medical inhalers. An alkali metal fraction in the glass of at most 15 wt % or even at most 6 wt % has emerged as being particularly advantageous.

According to one advantageous embodiment of the invention, the dielectric material of the evaporator comprises a glass. Having emerged as being particularly advantageous is a borosilicate glass, more particularly having the following constituents:

| | |
|---|---|
| SiO₂ | 50 to 85 wt % |
| B₂O₃ | 1 to 30 wt % |
| Al₂O₃ | 1 to 30 wt % |
| ΣLi₂O + Na₂O + K₂O | 0 to 30 wt % |
| ΣMgO+ CaO + BaO + SrO | 1 to 40 wt %. |

Other glasses, however, may also be used as dielectric material. Hence, as well as borosilicate glasses, bismuth glasses or zinc glasses, for example, have also emerged as being suitable. By the latter glasses or similar glasses with different oxides is meant that they comprise corresponding oxidic components—that is, for example, Bi₂O₃ or ZnO—as a key constituent, to an extent, for example, of at least 50 wt % or even up to 80 wt %.

Through the selection of the respective dielectric material, more particularly a glass, it is also possible to influence the thermal expansion behavior of the dielectric component. A low thermal expansion of said component in an evaporator application is advantageous here with regard to cyclical temperature stability or under cyclical temperature loading of the sintered body. In the context of the use of the composite in an electric cigarette, for example, this may occur as a result of repeated, often fairly short, heating cycles.

In a similar way as for the electrically conductive materials, the inertness or chemical stability of the glass is also relevant with regard, for example, to possible reactions, and/or their avoidance, between glass and electrically conductive material; this also applies, in particular, during the operation of producing a sintered body by thermal treatment, such as during the sintering procedure, for example. Inertness of the dielectric material toward the auxiliaries used in the production process, as for example toward sinter aids or pore formers, is also advantageous. When the sintered body is used, for example, as an evaporator or as a component in an evaporator, a high chemical stability or low reactivity of the glass relative to the substances to be evaporated, examples being propylene glycol, glycerol, water and/or mixtures thereof, and/or adjuvants therein, is essential. Preference is given to using glasses having a high chemical resistance, more particularly to glasses having a class 3 water resistance, more preferably glasses having a class 1 or 2 water resistance (measured according to ISO 719). Having additionally emerged as being advantageous in terms of their chemical resistance are glasses having a low fraction of network modifiers and/or having a high fraction of network formers. According to one embodiment, the glass has a fraction of network formers of at least 50 wt %, preferably a fraction of network formers of at least 70 wt %. Network formers are understood more particularly to be glass components which contribute to the formation of oxygen bridges in the glass, examples being SiO₂, B₂O₃ and Al₂O₃.

As dielectric materials it is alternatively possible to use glass-ceramics, ceramics or plastics, provided they can be processed below the melting temperature of the electrically conducting material used.

A glass-ceramic in the sense of the present disclosure is understood here to be the transformation product of a green glass, i.e., of a crystallizable glass, by heating to appropriate temperatures under which ceramization takes place. This glass-ceramic comprises both a glassy phase and crystallites.

In the case of the use of ceramics as a dielectric material with usually high melting temperatures, especially when this temperature is situated above those of the metals employed, sintering promoters, such as a glass, preferably a glass described above, are added, so that a sintered body is sintered or sinterable by means of liquid-phase sintering with formation of a liquid phase of this same glass.

According to one embodiment of the invention, the sintered body comprises a mixture of at least two different dielectric materials. The dielectric fraction of the sintered body in this case represents a composite comprising each of the dielectric materials used. More particularly it may be a composite of glass and ceramic. In contrast to the glass-ceramic, the composite is an assembly of materials.

It has emerged here are being particularly advantageous if at least one of the dielectric components is a glass and is preferably not below a fraction of 5 vol % of the dielectric materials. Alternative dielectric components may be glass-ceramics, ceramics or plastics, provided they can be processed below melting temperature of the electrically conducting material used. In the case of embodiments in which the dielectric material comprises ceramics, it is necessary to take account of the usually high sintering temperature required for the ceramics. Where ceramics are used as a dielectric material, therefore, especially if the sintering temperature of the ceramic lies above the melting temperature of the metals, sintering promoters are added, so that a sintered body is sintered or sinterable by means of liquid-phase sintering with formation of a liquid phase of the sintering promoter. Suitable sintering promoters include, in particular, glasses, and especially here the glasses described above. In this case the ceramic fraction is at least 80 vol %, preferably at least 90 vol %, most preferably at least 95 vol %, based on the envisaged volume fraction of the dielectric material.

In one embodiment of the invention, the fraction of the ceramic as a proportion of the overall dielectric material in the sintered body is at least 50 vol %, preferably at least 75 vol % and especially preferably at least 90 vol %. Sintered bodies whose dielectric material is completely ceramic or at least almost completely ceramic are also possible without departing from the invention.

In the case of sintered bodies which overall have a fairly low fraction of dielectric material, however, it may be advantageous, in terms of sintering processability and of the mechanical stability of the sintered body, if at least 50 vol % of the entire dielectric material, more particularly at least 70 vol % of the dielectric material, is a glass. This is advantageous in particular in the case of sintered bodies having a total fraction of dielectric material of less than 25 vol %, more particularly of less than 15 vol % of the sintered body.

As well as promoting the sinterability, the glass fraction, which in that case is substantially melted, also makes a positive contribution to the capacity for such a sintered body to be coated with ceramic fraction of the dielectric material. In this case it is possible to tailor the particle sizes of ceramic and glass to one another in such a way as to prevent segregation or separation of the powders or agglomeration of a powder owing to greatly different particle sizes during production. It has emerged as being advantageous in this context not to choose a greater particle size for the glass than for the ceramic fraction. Bimodal or polymodal distributions in terms of the particle size distributions of glass fraction and ceramic fraction are also possible and in certain cases allow the particle sizes of all the materials to be adapted to one another. When glass-ceramics are used for producing a sintered body comprising a glass-ceramic, the addition of a volume fraction of glass or the replacement of a volume fraction of the glass-ceramic by a glass may also be advantageous in terms of the sinterability of the workpiece.

In a further variant, further materials may be added to a mixture of electrically conductive and dielectric materials in order, for example, to influence the processing or production of a sintered body. Hence, in particular, so-called sinter aids may be used for modifying the sintering conditions, such as adjusting, in particular lowering, the processing temperature, for example, and/or materials which permit modification to properties of the sintered body. Hence, especially when using high-melting ceramics as a dielectric material, the addition of a sintering promoter, such as a glass, advantageously an above-described glass, allows sintering to take place with formation of a liquid phase at temperatures at which the electrically conductive material does not melt. In this way, furthermore, for example, the thermal conductivity may be made adjustable in relation to thermal isolation versus heating power, heating rate or heating-up of surrounding components, of an E-cigarette, for example, or else the surface properties of the sintered body, in terms of absorption, desorption and/or continued flow of media for evaporation.

Moreover, the corresponding dielectric materials ought in principle to have sufficient chemical resistance and also resistance toward water and the constituents of liquids for evaporation, such as propylene glycol and glycerol, for example, but also to the metals. Examples of suitable plastics include temperature-stable polymers such as polyetheretherketone (PEEK), polyetherketoneketone (PEKK) or polyamides (PA).

According to one embodiment of the invention, the evaporator has a mechanical electrical contacting, an electrical contacting through an electrically conducting connector, or a substance-to-substance electrically conducting bond. The electrical contacting is accomplished preferably through a soldered bond.

In one variant of the invention, the sintered body additionally has an electrically conductive coating. Having proven particularly advantageous in this context is an electrically conductive coating which extends over the entire surface of the sintered body. Hence even the surfaces of the sintered body that are formed by the pore surfaces in the interior of the sintered body are provided with the electrically conductive coating. This is particularly advantageous since therefore the coated sintered body as well has a uniform electrical conductivity. Examples which have emerged of coating materials include indium tin oxide (ITO) and aluminum-doped zinc oxide (AZO). It is also possible to use coatings which generally comprise at least one of these materials.

The additional coating, which depending on coating process may also be applied only partially or in portions on a sintered body, allows the electrical conductivity of the evaporator to be modified without any alteration in the composition of the sintered body. Hence according to one embodiment the electrical conductivity of the sintered body may be adapted or adjusted by the coating, more particularly increased and/or homogenized. This may be utilized for the purpose, for example, of generating evaporators having particularly high electrical conductivities, by coating sintered bodies with a relatively high amount of electrically conductive material. This also makes it possible to establish a required electrical conductivity based on mandated basic conductivities of sintered bodies as composites composed of dielectric material and electrically conductive material, by application of suitable layer thicknesses of the coating. In this way, likewise, any fluctuation in a conductivity of the sintered body or in its basic conductivity can be easily evened out. Moreover, in particular through local and/or lateral structuring of the electrically conductive coating, it is possible to realize a composite having a locally adapted conductivity, by means of local limitation of the conductivity, for example. Through lateral structuring of the coating on the sintered body it is therefore possible to obtain zones having different electrical conductivities. In this way, for example, the sintered body can be divided into local heating zones and/or storage zones. The controlled establishment of transport zones and transport pathways may also take place in this way.

Additionally, by means of a coating, it is also possible to influence the surface properties, examples being the surface activity or surface energy, of the sintered body or evaporator, in order, for example, to alter or adjust the take-up, transport and delivery or evaporation of a liquid. The inertness of the sintered body may also be further improved by subjecting it, so to speak, to passivation by means of a coating, hence in order to protect it, for example, in particular in operation, from corrosion, degradation or aging as a result of reaction with air or with liquid for evaporation. Thermomechanical properties of the sintered body may also be adapted, improved or adjusted, such as the mechanical strength and/or the thermal conductivity, for example. In this case, however, one coating may also address one or more of these properties.

In another embodiment, the sintered body contains only a relatively low fraction of electrically conductive material, in particular in the range from 5 to 15 vol %, and therefore has a relatively low electrical conductivity. The latter can be increased by application of an electrically conductive coating. Because the sintered body already has electrical conductivity, only relatively low layer thicknesses are necessary relative to the coating of sintered bodies which do not comprise electrically conductive material. In comparison with a sintered body which is composed of a purely dielectric material, in the case of the sintered body of the invention, in accordance with its basic electrical conductivity, the amount of coating material needed can be reduced, by up to 90%, for example, in order to attain comparable electrical conductivities. According to a further embodiment, on the basis of a very low fraction of electrically conductive material and/or of the electrically conductive material used, the composite has no conductivity or only a very low conductivity, such that the individual electrically conductive particles in the sintered body have little or no crosslinking. Through application of the above-described, electrically conductive coating, the electrically conductive particles are connected to one another and an electrically conductive coated sintered body is obtained. For this purpose, as compared with a sintered body without electrically conductive material, only relatively little coating material is needed in order to achieve sufficient electrical conductivity.

The mean layer thickness of the electrically conductive coating is preferably less than 10 μm or even less than 1 μm, down to a few nanometers or a few 10s of nm. The necessary or possible layer thickness here is determined mainly by its nature and the method in which the coating is produced. Hence ITO coatings are available in an electrical conductivity range from a few 10⁴ S/m to a few 10⁶ S/m, and a TiN coating from a few S/m to a few 10⁻³ S/m. One of the effects of these low layer thicknesses is that only little coating material is needed. At the same time there is a marked reduction in the risk of smaller pores becoming clogged by the coating and therefore no longer being available as evaporation volume. The required or sufficient layer thickness here is dependent on the electrical conductivity of the layer material. The attainable or achievable layer thickness is also dependent on the methods of coating, e.g., by means of liquid or vapor deposition, or electrochemically. With such methods, then, layers are applied, preferably densely and homogeneously, to a sintered body in order to establish its requisite electrical conductivity and its required operational heating behavior, uniformly or else locally limited in volume, for example, of the sintered body.

The evaporators of the invention are suitable in particular for use as a component in an electronic cigarette, a medical inhaler, a fragrance dispenser or a room humidifier. In this case, for example, the evaporator may also be used for the indirect evaporation of liquids or solids, such as waxes or resins, for example. In one refinement of the invention, accordingly, air or gas flows through the sintered body, which heats it. A possible use of this refinement lies in medical inhalers. Use as a radiant heater is also possible.

A further aspect of the invention lies in the provision of a method for producing an evaporator. The method of the invention in this case comprises at least the following method steps a) to d):
a) providing an electrically conducting material and a dielectric material in powder form,
b) mixing the powders provided in step a), preferably optionally with a pore former,
c) generating a green body from the powder mixture provided in step b) by pressing, casting or extruding, and
d) sintering the green body generated in step c).

Here in particular in the case of plastics as dielectric material, steps c) and d) may also take place in parallel (simultaneously) or sequentially in one assembly, for example an extruder or in injection molding, for example, possibly also encompassing the step b). Such methods are in principle also applicable to the other dielectric materials, but frequently costly and complicated and less readily controllable. The concept of sintering is also understood here as an operating step leading to the solidification of such a body.

The fraction of the electrically conductive material in the overall materials provided in step a) is not more than 90 vol % here. According to one preferred embodiment, the fraction of electrically conductive material is in the range from 5 to 70 vol %, preferably in the range from 10 to 60 vol % and more preferably in the range from 15 to 40 vol %. Dielectric material provided in step a) comprises glass, crystallizable glasses, glass-ceramic, ceramics or plastics or mixtures thereof in powder form.

According to one embodiment of the invention, the fraction of dielectric material in the materials provided in step a) is at least 10 vol %, preferably 30 to 95 vol %. The dielectric material here has a lower softening point or melting point than the electrically conductive material.

From the mixture provided in step b), a green body is produced in a subsequent step c). This may be accomplished, for example, by pressing or extruding operations or by a casting operation. In one embodiment of the invention, a slip is produced from the mixture provided in step b) and is subsequently cast.

In step d) the green body is sintered. The sintering temperature here corresponds at least to the softening temperature of the dielectric material, and so the sintering operation causes the dielectric material to form a coherent matrix. At the same time, however, the sintering temperature is lower than the melting temperature of the electrically conductive material, and so the particle structure of the electrically conductive material is at least largely maintained. It has emerged that a combination of dielectric and electrically conductive materials wherein the dielectric material can be softened or processed at a temperature which is at least 10° C. or even at least 100° C. below the melting point of the electrically conductive material is particularly advantageous. As a result, in step d), the sintering can take place at a temperature which allows a sintered body with high mechanical strength. At the same time, however, it is ensured that dimensional stability of the electrically conductive particles in the sintered body and hence also the electrical conductivity of the sintered body are unaffected by the sintering operation. According to one embodiment of the invention, the sintering of the green body in step d) takes place at a sintering temperature in the range from 350 to 1000° C.

The sintered bodies produced with the method of the invention have a high mechanical stability, and so the sintered body can be reworked, for surface working or shaping, for example. According to one refinement of the invention, the sintered body, in a step e) downstream of step d), is ground, drilled, polished, milled and/or turned.

Electrical contacting of the sintered body, moreover, may take place in a step f) of the sintered bodies downstream of steps d) and/or e). Contacting by the application of an electrically conductive paste has proved to be particularly advantageous in this case.

According to one embodiment, the dielectric material provided in step a) has a thermal stability with respect to temperatures of at least 300° C. or even at least 400° C. In a refinement of the invention, a glass is provided as dielectric material in step a). In an embodiment of the invention, the glass provided in step a) has a transition temperature Tg in the range of more than 300° C., more particularly in the range from 500 to 800° C. As a result it is possible in step d) to sinter at sintering temperatures which ensure the dimensional stability of the electrically conductive particles. At the same time, however, the transition temperature of the glass is well above the operating temperature of the evaporator.

In an embodiment of the invention, a glass having an alkali metal content <15 wt % or even <6 wt %, or even an alkali-free glass, is provided in step a). Such glasses exhibit high mechanical strength and good chemical and thermal stability and have no reaction or virtually no reaction with the electrically conductive materials even at high temperatures. A borosilicate glass is preferably provided as dielectric material in step a).

It has emerged as being particularly advantageous if the electrically conductive particles provided in step a) have a mean particle size in the range from 0.1 to 1000 μm, preferably in the range from 1 to 50 μm.

Alternatively or additionally, the particles of the dielectric material provided in step a) have a mean particle size in the range from 1 to 50 μm. More particularly the mean particle size of the dielectric material is less than 30 μm. Such particle sizes of the dielectric material lead to sintered bodies in which the maximum distance between adjacent electrically conductive particles is less than 30 μm or even less than 10 μm. In the corresponding sintered body, this ensures conduction of current even when amounts of electrically conductive material are low.

In step b), a particularly homogeneous mixture can also be obtained by harmonizing the particle sizes of the powders of dielectric and electrically conductive material with one another in such a way that there is no segregation or separation of the powders or agglomeration of a powder, owing to greatly differing particle sizes. A homogeneous mixture in step b) is also advantageous for the homogeneity of the composite and hence for the homogeneity of the electrical conductivity as well. Furthermore, particle sizes of the powders or of one powder that are too small, even if they are harmonized with one another in terms of the particle sizes, are to be avoided as far as possible, in order to minimize unnecessary dusting during the processing of the powders.

Electrically conductive materials provided in step a) are preferably precious metals, aluminum, copper, tungsten, molybdenum, chromium, nickel, titanium, titanium nitride, iron, stainless steel, silicon and/or alloy or mixtures thereof and/or carbon, preferably in the form of graphene or graphite or nanotubes or nanorods. Electrically conductive materials provided are preferably gold particles, silver particles or platinum particles. These materials here in particular have not only high electrical conductivities but also high chemical stability and/or high melting points.

According to one refinement of the invention, the particles of the electrically conductive material provided in step a) have a platelet-shaped geometry, preferably a platelet-shaped geometry with a maximum thickness dₘax and a maximum length lₘₐₓ, for which dₘₐₓ<lₘₐₓ. Such geometries are especially suitable for use in sintered bodies with a low fraction of electrically conductive materials, i.e. in sintered bodies where current flow is realized to a large extent through electron tunneling currents. In this case, in particular platelet-shaped particles whose maximum length is at least twice as great as the maximum width have emerged as being advantageous. According to one preferred embodiment, the ratio of maximum thickness to the maximum length is 1:2 to 1:7.

In a refinement of the invention, an electrically conductive coating, more particularly a coating, very preferably an oxidic ITO or AZO or nitridic, more particularly TiN-containing, or metallic coating, is applied to the sintered body in a step g) downstream of step d) and/or step e). In one preferred embodiment here, the coating is applied by means of sol-gel methods or CVD methods to the surface of the sintered body. It is likewise conceivable, especially since the sintered body already has at least a basic conductivity, to contemplate layer materials which can be processed and/or applied electrochemically, examples being gold, silver or copper and/or combinations thereof, as a layer sequence, for example.

### BRIEF DESCRIPTION OF THE DRAWINGS

The invention is described in more detail below with reference to exemplary embodiments and figures, in which:

FIG. 1 shows a schematic representation of a conventional evaporator,

FIG. 2 shows a schematic representation of a sintered body with electrical contacting on the lateral faces of the sintered body,

FIG. 3 shows a schematic representation of one embodiment of an evaporator of the invention,

FIG. 4 shows a schematic representation of one embodiment of a sintered body of the invention in cross section,

FIG. 5 shows an enlarged detail of the cross section shown in FIG. 4, and

FIG. 6 shows an SEM micrograph of an exemplary embodiment, and

FIG. 7 shows a schematic representation of a further exemplary embodiment with an additional electrically conductive coating on the sintered body.

### DETAILED DESCRIPTION

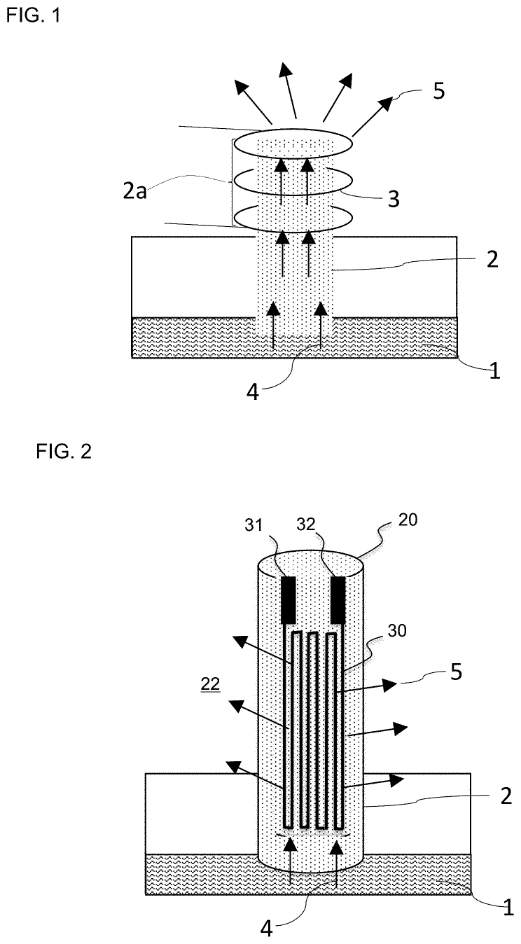

FIG. 1 shows an example of a conventional evaporator with a porous sintered body 2 as liquid reservoir. As a result of the capillary forces of the porous sintered body 2, the liquid 1 for evaporation is taken up by the porous sintered body 2 and transported further in all directions of the sintered body 2. The capillary forces here are symbolized by the arrows 4. In the upper portion of the sintered body 2, a heating coil 3 is positioned such that the corresponding portion 2a of the sintered body 2 is heated by thermal radiation. The heating coil 3 is therefore brought very close to the lateral faces of the sintered body 2 and is intended as far as possible not to contact the lateral faces. In practice, however, direct contact between heating wire and lateral face is often unavoidable.

In the heating region 2a, the liquid 1 is evaporated. This is represented by the arrows 5. The rate of evaporation here is dependent on the temperature and on the ambient pressure. The higher the temperature and the lower the pressure, the quicker the liquid evaporates in the heating region 2a.

Since the evaporation of the liquid 1 takes place only locally on the lateral faces of the heating region 2a of the sintered body, this local region must be heated with relatively high heating powers in order to achieve rapid evaporation within from 1 to 2 seconds. It is therefore necessary to apply high temperatures of more than 200° C. High heating powers, particularly in a narrowly locally confined region, however, may lead to local overheating and may therefore lead possibly to a decomposition of the liquid 1 for evaporation and of the material of the liquid reservoir and/or wick.

Furthermore, high heating powers may also lead to excessively rapid evaporation, so that the capillary forces are unable to provide further liquid 1 for evaporation quickly enough. This likewise leads to an overheating of the lateral surfaces of the sintered body in the heating region 2a. It is therefore possible to install a unit, as for example a voltage, power and/or temperature adjusting, controlling or regulating unit (not represented here), although this is at the expense of the battery life and limits the maximum evaporation quantity.

Disadvantages of the evaporator represented in FIG. 1 and known from the prior art are therefore the local heating method and the associated ineffective heat transport, the complex and expensive control unit, and the risk of overheating and decomposition of the liquid for evaporation and of the reservoir/wick material.

FIG. 2 shows an evaporator unit known from the prior art in which the heating element 30 is disposed directly on the sintered body 20. More particularly the heating element 30 is firmly connected to the sintered body 20. A connection of this kind may be achieved in particular by designing the heating element 30 as a film resistor. For this purpose, an electrically conducting coating with a ladder-like structuring is applied in the manner of a film resistor to the sintered body 20. One of the advantages of a heating element 30 in the form of a coating applied directly on the sintered body 20 is that of achieving effective heat contact, which enables rapid heating. However, the evaporator unit shown in FIG. 2 also has only a locally limited evaporation surface, and so here as well there is a risk of overheating of the surface.

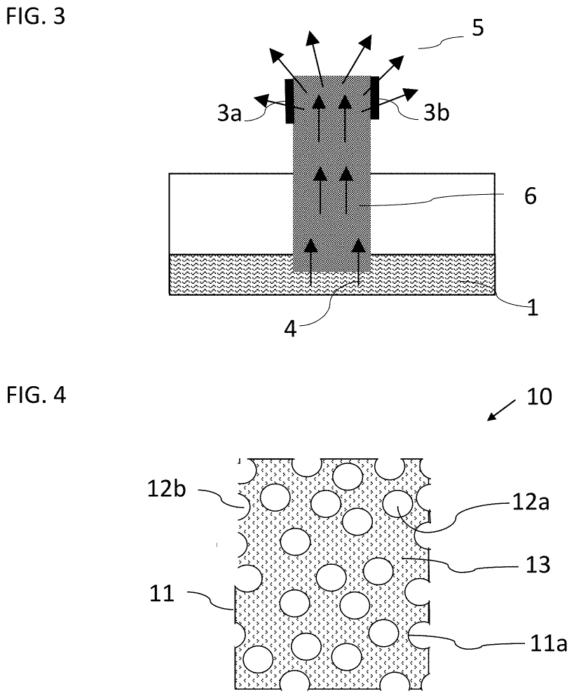

FIG. 3 shows schematically the construction of an evaporator with a sintered body 6 of the invention. As for the porous sintered body 2 in FIG. 1 and FIG. 2, this sintered body 6 is immersed in the liquid 1 for evaporation. Capillary forces (represented by the arrows 4) bring about transport of the liquid for evaporation into the entire volume of the sintered body 6. Therefore, when an electrical voltage is applied between the contacts 3a and 3b, the sintered body 6 is heated in the entire volume region between the contacts 3a and 3b of high surface area. In contradistinction to the evaporator shown in FIG. 2, therefore, the liquid 1 is formed not only on the lateral faces of the sintered body, but rather in the entire volume region between the electrical contacts of the sintered body 6. There is therefore no need for capillary transport to the lateral faces or heated faces or elements of the sintered body 6. Moreover, there is less risk of a local overheating. Since the evaporation in the volume operates with substantially greater efficiency than by means of a heating coil in a locally limited heating region, the evaporation can take place at substantially lower temperatures and with a lower heating power. A lower electrical power requirement is advantageous in that it thus increases the usage time per secondary cell charge and/or allows smaller secondary cells or batteries to be installed.

FIG. 4 shows a schematic representation of a cross section through a sintered body 10 as an exemplary embodiment of the invention. This sintered body 10 comprises a composite material 11 and pores 12a and 12b distributed therein. The composite material 11 has an electrical conductivity in the range from 0.1 to 105 S/m. Where a voltage is applied to the sintered body 10, current flows through the entire volume of the sintered body 10, which is heated accordingly.

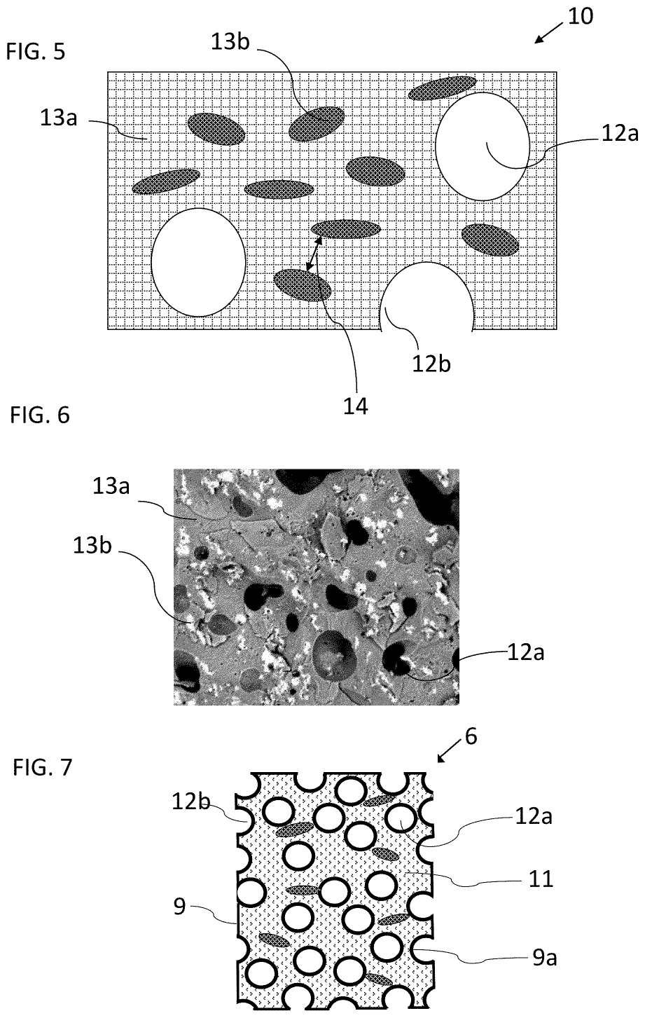

FIG. 5 represents a detail of the sintered body 10, in enlarged form. The composite material 11 is formed by a dielectric matrix 13a and also by electrically conductive particles 13b distributed homogeneously in the matrix 13a. In the embodiment shown in FIG. 5, the electrically conductive particles 13b have a platelet-shaped geometry. A corresponding sintered body 6 as example 1 with an electrical conductivity in the range from 1 to 5 S/m and a porosity of around 30 vol % can be obtained here according to operating steps a to d, by first providing a mixture of 50 vol % each of a glass and titanium, with a particle size d₅₀ selected from the range from 20 to 50 μm and an elongate particle morphology, producing a green body from this mixture, and subsequently sintering this green body by thermal treatment in a regular kiln atmosphere at a temperature which corresponds approximately to the softening temperature of the glass employed, in this case around 700° C., for 20 min-120 min to form the sintered body 6.

When using a further glass with a softening temperature about 200° C. higher on the part of the glass employed, accordingly, it is possible to obtain, on sintering at around 920 to 940° C. for 20 min to 120 min, a sintered body 6 as example 2 with an electrical conductivity in the range from 1 to 10 S/m.

Here and in examples below, unless noted otherwise, the electrical conductivity is ascertained by resistance measurement on, for example, specimens of around 5 to 10 mm in diameter and 5 to 10 mm in height and by conversion of the resistance value into electrical conductivity, with the measurement tips being mounted or arranged mechanically, manually at the opposite diameters, without further auxiliaries (for example, conductive paste or soldering-on of contacts). From these examples 1 and 2 it is clear here that the dielectric material, in this case the type of glass used, exerts only a moderate influence over the electrical conductivity of the sintered body. Instead, the electrical conductivity is critically determined by the nature of the electrically conductive material and the amount thereof in the sintered body.

In another refinement, the dielectric material, in accordance for instance with examples 1 and 2, is modified such that the dielectric fraction of the sintered body contains not only glass but also ceramic. Hence the ceramic fraction in the dielectric material may be for example up to 97 vol %. Hence in the case of sintered bodies with a ceramic fraction of 97 vol % (based on the dielectric fraction), for example, it is likewise possible to obtain electrical conductivities in the range from 1 to 10 S/m. Sintered bodies which, conversely, have only a low ceramic fraction in the dielectric material likewise exhibit comparable electrical conductivities. The inventors therefore suppose that the nature of the dielectric material used, while influencing the mechanical properties, has only a very low influence on the electrical conductivity of the sintered body. The same is true of sintered bodies whose dielectric fraction contains a mixture of glass-ceramic with one or both of the glass and ceramic constituents. A glass-ceramic fraction may be formed here also by the inclusion in the green body of a crystallizable glass which on sintering at a corresponding temperature for ceramization of this glass undergoes ceramization and is then present as a glass-ceramic. Below such a temperature, a crystallizable glass remains in the glassy state.

Moreover, sintered bodies 6 as example 3 with an electrical conductivity in the range from 100 to 1000 S/m for a porosity of around 55 vol %, may be obtained according to operating steps a to d by the provision first of a mixture of 85 vol % each of a glass and 15 vol % of silver, with a particle size d₅₀ of 15 to 20 μm and an elongate particle morphology, the production therefrom of a green body, and the subsequent sintering of this green body by thermal treatment in a regular kiln atmosphere at a temperature which corresponds approximately to the softening temperature of the glass employed, here around 930 to 950° C., for 20 min-120 min to form the sintered body 6. When using a different particle morphology on the part of the silver employed, in the present case round particle morphology with d₅₀ of likewise 15 to 20 μm, accordingly, sintered bodies 6 as example 4 are obtained with an electrical conductivity in the range from 0.5 to 1 S/m. This highlights the influence of the particle shape of the electrically conducting material on the electrical conductivity.

Sintered bodies 6 as example 5 or 6 with a porosity of around 55 vol % and a conductivity of around 1500 S/m may be obtained by means of mixtures of 70 vol % of glass with 30 vol % of molybdenum (d₅₀ of 1 to 3 μm) or of a mixture of 70 vol % of glass with 30 vol % of tungsten (d₅₀ of 1 to 2 μm), by thermal treatment in regular kiln atmosphere at a temperature which corresponds approximately to the softening temperature of the glass employed, here around 900 to 950° C., for 20 min-120 min. In this case the resistance of the specimens was measured on their two opposite diameters with the aid of conductive paste applied there.

FIG. 6 shows an SEM micrograph of a cross section through a sintered body of the invention, as a further exemplary embodiment. Here the electrically conductive particles 13b appear as light-colored structures in the dielectric material 13a. The pores 12a have a primarily round cross section. The cross-sectional geometry of the pores 12a is determined by the particle geometry of the pore former used in the production method.

FIG. 7 shows the construction of a coated sintered body 6 with open porosity by means of a schematic cross section through a further exemplary embodiment. The coated sintered body 1 comprises a porous matrix of composite material 11 with open pores 12a, 12b. Some of the open pores 12b with their pore surface form the lateral faces of the sintered body, while another set of the pores 12a form the interior of the sintered body. All of the surfaces of the sintered body have an electrically conductive coating 9a, in the form of an ITO coating, for example. When a voltage is applied on the sintered body, the current flows through the entire volume of the sintered body.

A correspondingly coated sintered body 6 as example 8 may be obtained in this context by first producing a glass-metal composite having a relatively low electrical conductivity in the range from 0.1 to 100 S/m, according to one of examples 1 or 4, for example. For this purpose it is also possible further, for example, to produce a sintered body from 95 to 86 vol % of borosilicate glass and 5-15 vol % of silver with elongate silver particles having a particle size in the range from 1 to 60 μm by sintering in air at a temperature in the sintering range of 900-950° C. for 20 min to 120 min. To obtain a desired electrical conductivity in the range from 100 to 600 S/m, the sintered body is provided subsequently with an electrically conductive coating, for example an ITO-containing or AZO-containing coating. As a result of the basic electrical conductivity of the sintered body, in this case (compared with a sintered body without electrically conductive material) less than 50% of the coating material is needed. Furthermore, the coating operation is also less time-intensive. Hence the operating time needed for the coating process can be reduced by up to 70%.

### LIST OF REFERENCE SIGNS

1 carrier liquid
2 sintered body
2a heating zone
3, 30 heating element
3a, 3b contacts
4 capillary forces
5 vapor
6 sintered body
8a, 8b pores
9, 9a electrically conductive coating
10 electrically conductive sintered body
11 composite material
12a, 12b pore
13a dielectric material
13b electrically conductive particles
14 distance between adjacent electrically conductive particles
sintered body
22 evaporator
31, 32 contacting

What is claimed is:

1. An evaporator comprising:
a porous sintered body formed by a composite of an electrically conductive material and a dielectric material, wherein the porous sintered body has an open porosity in a range from 10 to 90% and an electrical conductivity in the range from 0.1 to 105 S/m,
wherein the dielectric material is selected from a group consisting of glass, glass-ceramic, ceramic, plastic, and combinations thereof, and
wherein the composite has a faction of the dielectric material of 10 to 95 vol % and a fraction of the electrically conductive material of not more than 90 vol %.

2. The evaporator of claim 1, wherein the electrical conductivity is in the range from 10 to 10000 S/m.

3. The evaporator of claim 1, further comprising an electrical resistance in a range from 0.05 to 5 ohms.

4. The evaporator of claim 3, wherein the electrical resistance is from 0.1 to 5 ohms.

5. The evaporator of claim 3, wherein the porous sintered body comprises the electrical resistance.

6. The evaporator of claim 1, further comprising a voltage in the range from 1 to 12 V and/or a heating output of from 1 to 500 W.

7. The evaporator of claim 1, wherein the electrically conductive material is selected from a group consisting of: tungsten, molybdenum, iron, titanium, aluminum, copper, chromium, nickel, precious metal, platinum, gold, silver, stainless steel, silicon, titanium nitride, graphite, and any mixture or alloys thereof.

8. The evaporator of claim 1, wherein the electrically conducting material has a feature selected from a group consisting of: a resistance with positive temperature coefficient, a temperature coefficient of resistance of at least −0.0001 l/K, a temperature coefficient of resistance of less than 0.008 l/K, and a temperature coefficient of resistance of at least −0.0001 l/K and less than 0.008 l/K.

9. The evaporator of claim 1, wherein the faction of the electrically conductive material is in a range from 15 to 40 vol %.

10. The evaporator of claim 1, wherein the faction of the electrically conductive material is in a range from 5 to 70 vol % and the sintered body further comprises an electrically conductive coating.

11. The evaporator of claim 10, wherein the electrically conductive coating is on internal surface of pores of the open porosity.

12. The evaporator of claim 1, wherein the electrically conductive material comprises particles having a feature selected from a group consisting of: a particle size d₅₀ in a range from 0.1 μm to 1000 μm, a particle size d₅₀ in a range from 1 to 200 μm, a particle size d₅₀ in a range from 1 to 50 μm, a shape that is platelet-shape, a maximum length that is larger than a maximum thickness, a maximum length that is larger than twice a maximum thickness, and a maximum length that is larger than seven times a maximum thickness.

13. The evaporator of claim 1, wherein the open porosity comprises pores having a mean pore size in a range from 1 μm to 5000 μm.

14. The evaporator of claim 1, wherein the electrical conductivity is in a range from >30 to 70 S/μm and the fraction of the electrically conductive material in the sintered body is 5 to 40 vol %.

15. The evaporator of claim 1, wherein the electrical conductivity in the range from 10 to 30 S/μm and the fraction of the electrically conductive material in the sintered body is 10 to 60 vol %.

16. The evaporator of claim 1, wherein the electrical conductivity in the range from 1 to <10 S/μm and the fraction of the electrically conductive material is 15 to 90 vol %.

17. The evaporator of claim 1, wherein the dielectric material comprises glass having a feature selected from a group consisting of: an alkali metal content ≤15% by weight, having an alkali metal content ≤6% by weight, a proportion of network formers of at least 50% by weight, a proportion of network formers of at least 70% by weight, a transformation temperature in a range from 300° C. to 900° C., a transformation temperature in a range from 500° C. to 800° C., a class 3 hydrolytic resistance measured in accordance with ISO 719, a class 2 hydrolytic resistance measured in accordance with ISO 719, and a class 1 hydrolytic resistance measured in accordance with ISO 719.

18. The evaporator of claim 1, wherein the dielectric material comprises glass comprising:

| | |
|---|---|
| SiO₂ | 50% to 85% by weight, |
| B₂O₃ | 1% to 30% by weight, |
| Al₂O₃ | 1% to 30% by weight, |
| ΣNa₂O + K₂O | 1% to 30% by weight, and |
| ΣMgO+ CaO + BaO + SrO | 1% to 40% by weight. |

19. The evaporator of claim 1, wherein the porous sintered body is configured as a component for a use selected from a group consisting of an electronic cigarette, a medical inhaler, a fragrance dispenser, a room humidifier, a disinfection device, and a gas heating device.

20. A method for producing an evaporator, comprising:
a) providing an electrically conducting material and a dielectric material in powder form;
b) mixing the electrically conducting material and the dielectric material provided in step a) with at least one pore former to produce a powder mixture;
c) generating a green body from the powder mixture provided in step b) by pressing, casting or extruding; and
d) sintering the green body generated in step c),
wherein the dielectric material comprises a material selected from a group consisting of glass, glass-ceramic, ceramic, and plastics, and
wherein the providing in step a) further comprises providing a fraction of the dielectric material from 5% to 70% by volume.
